(19) United States
(12) Patent Application Publication
(10) Pub. No.: US 2012/0041441 A1
(43) Pub. Date: Feb. 16, 2012

(54) CABLE TIE SYSTEM FOR STABILIZING BONE

(75) Inventors: Oren S. Bernstein, Portland, OR (US); Alexander R. Legg, Lebanon, OR (US)

(73) Assignee: ACUTE INNOVATIONS, LLC., Hillsboro, OR (US)

(21) Appl. No.: 13/205,428

(22) Filed: Aug. 8, 2011

Related U.S. Application Data
(60) Provisional application No. 61/372,416, filed on Aug. 10, 2010.

(51) Int. Cl. A61B 17/82

(52) U.S. Cl. 606/74

(57) ABSTRACT

A cable tie system, including methods, apparatus, and kits, for stabilizing bone. The system may provide a tie device comprising a strap including a leading region and a trailing region, a needle connected to the leading region of the strap, and a buckle connected to the trailing region of the strap. The buckle may be configured to receive the leading region of the strap such that the buckle and the strap collectively form a loop and a ratchet mechanism. The tie device also may comprise a handle connected to the buckle, to allow a person to tension the loop around bone by pulling the strap and the handle in opposite directions. The handle may be detached from the tie device after use.

### CROSS-REFERENCE TO PRIORITY APPLICATION

This application is based upon and claims the benefit under 35 U.S.C. §119(e) of U.S. Provisional Patent Application Ser. No. 61/372,416, filed Aug. 10, 2010, which is incorporated herein by reference in its entirety for all purposes.

### INTRODUCTION

The rib cage, or thoracic cage, is composed of bone and cartilage that surround the chest cavity and organs therein, such as the heart and the lungs. In humans, the rib cage typically is consists of 24 ribs, twelve thoracic vertebrae, the sternum (or breastbone), and the costal cartilages. The ribs articulate with the thoracic vertebrae posteriorly and, with the exception of the bottom two pairs of ribs (the floating ribs), are connected to the sternum anteriorly via the costal cartilages.

Major surgery inside the chest cavity, such as open heart surgery, generally requires that the rib cage be opened. The most common procedure for opening the rib cage is for a surgeon to perform a median sternotomy by cutting through the sternum along its entire length, from top to bottom. The surgeon then urges apart the resulting sternal halves, to divide or “crack” the sternum, which forms a passageway to the chest cavity. After surgery in the chest cavity, the passageway is closed by returning the sternal halves to their original positions and then the sternal halves may be secured to one another using wire or cable sutures.

The surgeon may wire the sternal halves together after median sternotomy using a cerclage procedure in which wire sutures encircle the sternum at spaced intercostal positions along the sternum. To install each suture, a wire may be attached to a curved needle that is used to guide the wire around (or through) each sternal half.

Each wire may be tensioned and secured to itself around the sternum. The most common procedure is to twist the ends of the wire together. With this simple approach, twisting the wire ends applies increasing tension to the wire and draws the sternal halves into engagement with one another, to provide secure bony approximation of the sternal halves.

The use of wires for sternal closure has a number of drawbacks. For example, wires may break during and/or after installation. Also, wires tend to dig into bone when tensioned sufficiently to secure the sternal halves against one another. Furthermore, wires often have sharp ends that can irritate soft tissue.

Cable ties (also termed zip ties) provide an alternative to wire sutures for cerclage fixation. A cable tie generally includes a strap that extends from a buckle. A leading end of the strap may be fed into the buckle to form a loop around bone. The cable tie is self-locking: the buckle permits advancement of the strap through the buckle but restricts strap withdrawal. Thus, a surgeon may pull on the strap to urge advancement of the strap through the buckle, to increasingly tension the strap around bone, without the need for a separate securing or locking procedure to prevent strap slippage.

Despite their conceptual attractiveness, surgical cable ties have not gained wide acceptance as alternatives to wire sutures for sternal fixation, because such cable ties have disadvantages for use on bone. For example, cable ties may be difficult to tighten sufficiently. Also, cable ties may twist about bone as they are being tightened, which may apply torsional shear forces to the bone that damage the periosteum. In addition, cable ties may tighten unevenly, and may be difficult to place and assemble around the sternum. Furthermore, cable ties may have a relatively high profile at the buckle, which may produce considerable discomfort for the patient.

Improved surgical cable ties are needed for use on bone.

### SUMMARY

The present disclosure provides a cable tie system, including methods, apparatus, and kits, for stabilizing bone. The system may provide a tie device comprising a strap including a leading region and a trailing region, a needle connected to the leading region of the strap, and a buckle connected to the trailing region of the strap. The buckle may be configured to receive the leading region of the strap such that the buckle and the strap collectively form a loop and a ratchet mechanism. The tie device also may comprise a handle connected to the buckle, to allow a person to tension the loop around bone by pulling the strap and the handle in opposite directions. The handle may be detached from the tie device after use.

### BRIEF DESCRIPTION OF THE DRAWINGS

FIG. 1 is a view of selected components of any exemplary cable tie system for stabilizing bone, with an exemplary tie device of the system being tensioned around a cut sternum by a surgeon, in accordance with aspects of the present disclosure.

FIG. 2 is a view taken from above the tie device of FIG. 1, with the device in an extended configuration before installation, in accordance with aspects of present disclosure.

FIG. 3 is a magnified view of the leading end of the tie device of FIGS. 1 and 2, particularly a needle thereof, taken generally at the region indicated by “3” in FIG. 2.

FIG. 4 is a cross-sectional view of the needle of FIG. 3, taken generally along line 4-4 of FIG. 3.

FIG. 5 is another cross-sectional view of the needle of FIG. 3, taken generally along line 5-5 of FIG. 3.

FIG. 6 is a cross-sectional view of the strap of FIG. 3, taken generally along line 6-6 of FIG. 3.

FIG. 7 is a view of a buckle and a handle of the tie device of FIG. 2, taken generally at the region indicated by “7” in FIG. 2, and illustrating an exemplary approach for placing a leading region of the strap into the buckle by twisting the strap, in accordance with aspects of the present disclosure.

FIG. 8 is a view of the buckle and the handle of FIG. 7, taken generally as in FIG. 7, but with the handle detached from the buckle, and with another embodiment of the leading region of the strap placed into the buckle by another exemplary approach, namely, transverse insertion of a neck portion of the strap into the buckle followed by longitudinal advancement of the strap, in accordance with aspects of present disclosure.

FIG. 9 is a longitudinal sectional view of the buckle and strap of FIG. 8, taken generally along line 9-9 of FIG. 8 and illustrating an exemplary ratchet mechanism that restricts withdrawal of the strap from the buckle, in accordance with aspects of present disclosure.

FIG. 10 is a fragmentary view of another exemplary embodiment of a tie device, in accordance with aspects of the present disclosure.

FIG. 11 is a fragmentary view of the tie device of FIG. 10 after formation of a loop with the strap and buckle and after separation of the needle and the handle from the strap and buckle, in accordance with aspects of present disclosure.

FIG. 12 is a view of selected components of the cable tie system of FIG. 1, with the tie device of the system assembled around a cut sternum and with a tensioning device of the system readied to urge a spacer under the buckle of the tie device, in accordance with aspects of the present disclosure.

FIG. 13 is a view of the cable tie system of FIG. 1, taken generally as in FIG. 12, but after urging the spacer under the buckle and before separation of a handle of the tensioning device from the spacer, in accordance with aspects of the present disclosure.

FIG. 14 is a longitudinal sectional view of the tie and tensioning devices of FIG. 13, taken generally along line 14-14 of FIG. 13.

FIG. 15 is a fragmentary sectional view of the tie and tensioning devices of FIG. 14, taken generally at the region indicated at “15” in FIG. 14.

FIG. 16 is a fragmentary view of yet another exemplary embodiment of a tie device, in accordance with aspects of the present disclosure.

FIG. 17 is an exploded, fragmentary view of the tie device of FIG. 16, taken with a handle detached from a buckle of the device, in accordance with aspects of the present disclosure.

FIG. 18 is a fragmentary view of still another exemplary embodiment of a tie device, in accordance with aspects of the present disclosure.

FIG. 19 is a fragmentary bottom view of still yet another exemplary embodiment of a tie device, in accordance with aspects of the present disclosure.

FIG. 20 is a cross-sectional view of the tie device of FIG. 19 secured to itself around a cut bone, in accordance with aspects of the present disclosure.

### DETAILED DESCRIPTION

The present disclosure provides a cable tie system, including methods, apparatus, and kits, for stabilizing bone. The system may provide a tie device comprising a strap including a leading region and a trailing region, a needle connected to the leading region of the strap, and a buckle connected to the trailing region of the strap. The buckle may be configured to receive the leading region of the strap such that the buckle and the strap collectively form a loop and a ratchet mechanism. The tie device also may comprise a handle connected to the buckle, to allow a person to tension the loop around bone by pulling the strap and the handle in opposite directions. The handle may be detached from the tie device after use.

The cable tie system disclosed herein may offer substantial advantages. For example, the system may have a lower profile, may be easier to manipulate manually and/or with a tool(s), may produce less bone damage during installation, may be easier to tension uniformly around bone, may be better adapted for use in surgery, and/or may provide tension adjustment without advancing the strap further through the buckle.

These and other advantage of the cable tie system are disclosed in the following sections: (I) overview of cable tie systems, (II) exemplary tie devices with opposing handles and a top-loading buckle, (III) exemplary ratchet mechanism, (IV) exemplary tie device with a break-off handle and needle, (V) tensioning device for a cable tie, (VI) methods and kits, (VII) cable tie construction, and (VIII) examples.

### I. OVERVIEW OF CABLE TIE SYSTEMS

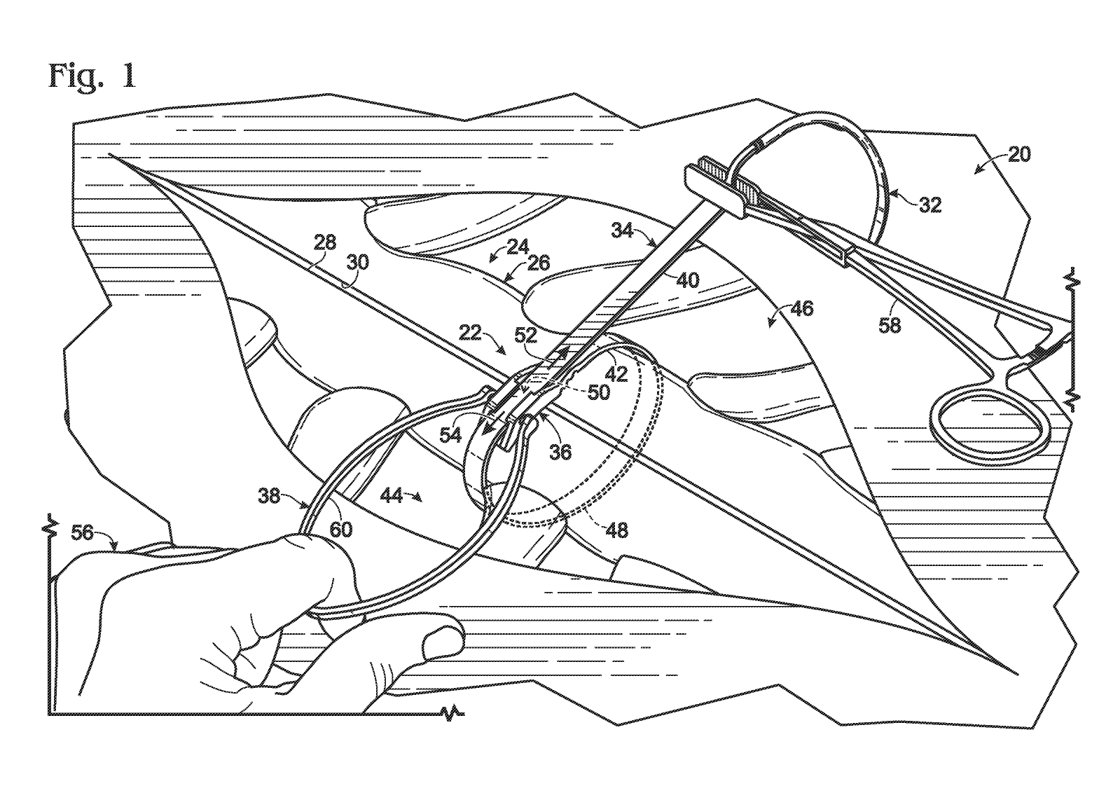

FIG. 1 shows selected components of any exemplary cable tie system 20 for stabilizing bone. The system may include an exemplary cerclage device, namely, a tie device 22 (also termed a cable tie), which may be secured to itself around bone 24, to hold portions of the bone together such that the bone is stabilized (e.g., fragments are bundled together, compressed, and/or fixed relative to one another). In the present illustration, cable tie 22 encircles a sternum 26 that has been cut longitudinally into two sternal halves or fragments 28, 30 via a median sternotomy. The cable tie may be tightened to compress bone surrounded by the cable tie. In any event, the cable tie may assist in stabilizing a cut or broken bone as the bone heals.

Cable tie 22 may include, in order along the device, a surgical needle 32, a strap 34, a buckle 36, and a buckle handle 38. The strap provides a leading region 40 and a trailing region 42. Leading region 40 may be connected, at a leading end of the strap, to needle 32. Trailing region 42 may be attached, at a trailing end of the strap, to buckle 36. Needle 32 may be used to thread leading region 40 of the strap around sternum 26 (e.g., through an opposing pair of intercostal spaces 44, 46 and under the sternum). Leading region 40 then may be loaded into buckle 36 to form a loop 48 that encircles the sternum. Buckle 36 may be disposed over the anterior surface or a lateral surface of the sternum.

The strap and buckle collectively may form a ratchet mechanism 50 that permits only unidirectional travel of the leading end of the strap through the buckle. In particular, the buckle may be configured to permit the strap to be advanced through (e.g., to slide through) the buckle in a forward (tightening) direction 52 to tighten the strap around the sternum (i.e., to decrease the width/diameter of loop 48). However, the buckle may be configured to lock the strap in place by restricting withdrawal of the strap in a reverse (loosening) direction 54 (e.g., to restrict the strap from sliding back out of the buckle). In other words, the buckle may restrict removal of the leading region of the strap from the buckle and/or may restrict loosening of the strap around the sternum (i.e., to increase the size of the encircling loop). Thus, the cable tie may provide locking engagement of the strap with the buckle to prevent loosening of the device about bone or bone portions. Moreover, the cable tie may be readily tensioned, or tightened, onto bone while preventing slippage or loosening.

FIG. 1 shows cable tie 22, and particularly strap 34, being tensioned around bone by a person (e.g., a surgeon 56) by advancement of leading region 40 of the strap through the buckle. The surgeon is applying opposing forces to the strap and the buckle, respectively. Leading region 40 of the strap may be grasped with a person's hand (e.g., with at least one finger (such as one or more fingers of a gloved hand)) or may be engaged with a tool, such as with gripping tool or pliers 58 (e.g., a needle driver), as shown here. A handle loop 60 formed by buckle handle 38 also may be grasped manually or gripped with a tool. In any event, the surgeon may pull the leading region of the strap in forward direction 52 and the buckle (via handle 38) in reverse direction 54, which advances the leading region of the strap in forward direction 52 through the buckle. Application of the opposing forces allows a surgeon to control the buckle position more precisely (e.g., restricting the cable tie from twisting about the bone), encourages application of a more uniform tension to the strap, and minimizes torsional shear exerted by the strap and/or buckle on the surface of the bone.

### II. EXEMPLARY TIE DEVICES WITH OPPOSING HANDLES AND A TOP-LOADING BUCKLE

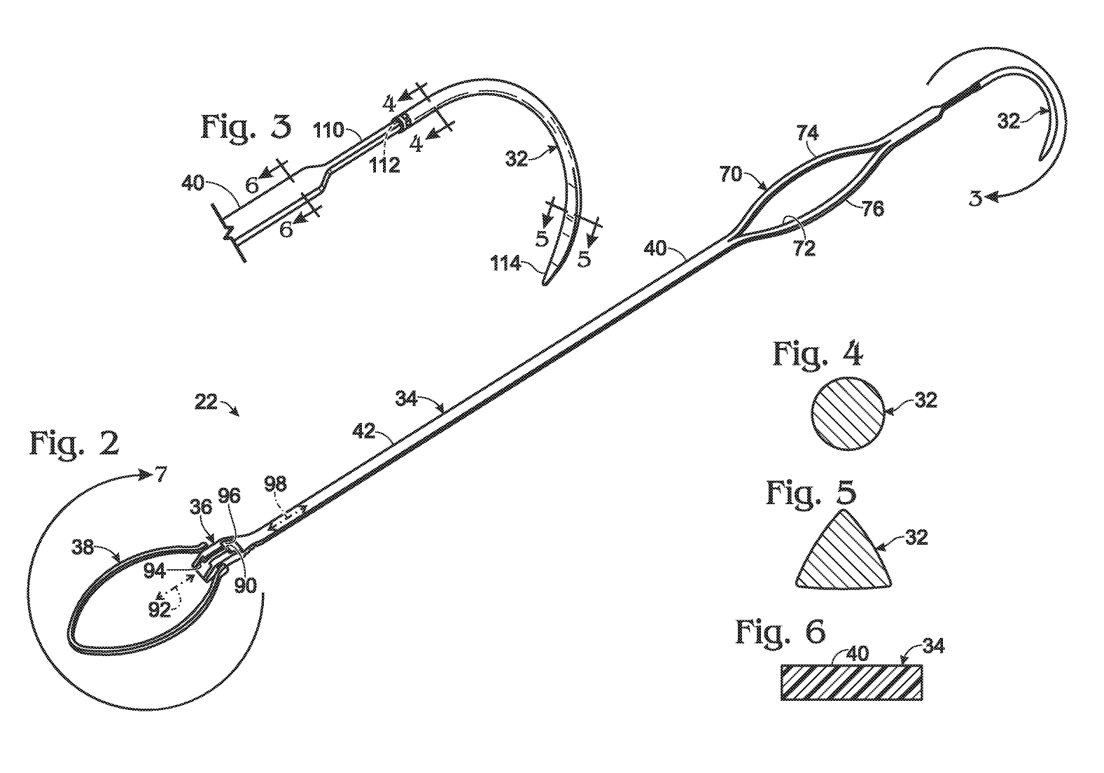

FIG. 2 shows cable tie 22 in an extended configuration before installation. The cable tie may have a low-profile design in which a minimum material thickness is utilized, and may have smoothly contoured surfaces for preventing damage to contacting or surrounding body tissue.

Strap 34 is elongate and provides leading and trailing regions 40, 42 having any suitable relative lengths and dispositions. The strap may be compliant, which allows the strap to conform at least generally to the cross-sectional shape of bone that the strap encircles. Leading region 40 is separated from buckle 36 by trailing region 42 (prior to loading into the buckle). Trailing region 42 is proximate to buckle 36 and extends from the buckle. The leading and trailing regions may be continuous with one another.

Strap 34 may form a strap handle 70 in leading region 40, with the handle sized to receive at least one finger of a person's hand. The strap handle may or may not be configured to be received by buckle 36. In some embodiments, the strap handle may be formed by a longitudinal slit 72 that separates a longitudinal portion of strap 34 into branches 74, 76. Handle 70 thus may be structured as a loop (e.g., a finger loop), which may be opened by separating branches 74, 76 and closed by bringing the branches together. Accordingly, handle 70 may be connected to a clamp, clip, band, or the like, which may be applied and/or adjusted to hold the branches together. With the branches held together, the width/cross-sectional size of strap handle 70 may be about the same as other regions of the strap. Closing strap handle 70 may be desirable when inserting the strap around bone, to minimize tissue damage, and/or when advancing the leading region of the strap through buckle 36. In some embodiments, slit 72 may be replaced by two or more shorter perforations that are arranged along the strap and that hold the branches together while the strap is placed around bone and through the buckle. The perforations then can be joined to form slit 72 by tearing/cutting intervening webs of material between the perforations.

Buckle 36 may define a channel or opening 90 (a passage) configured to receive leading region 40 of strap 34. The channel permits the strap received in the channel to move in one direction through the buckle along a channel axis or travel axis 92. Channel 90 extends from an entrance end 94 (a receiving end for end-loading the strap into the buckle) to an exit end 96. Travel axis 92 may be at least substantially parallel to a longitudinal axis 98 defined locally by trailing region 42 of the strap adjacent the buckle, as shown here, to provide an “in-line” configuration of the strap and buckle. In other words, the leading region of the strap may extend through the buckle and the trailing region of the strap may extend from the buckle substantially parallel to each other. For example, travel axis 92 may be within about 30, 20, or 10 degrees from parallel to longitudinal axis 98. Thus, when successive portions of the leading region of the strap are fed through the channel, such that the leading region extends beyond the exit end of the channel, a portion of the leading region may extend parallel to and overlie the trailing region of the strap adjacent the buckle. This in-line configuration may be advantageous to minimize the profile of the strap and buckle above bone. In other examples, the leading region may extend through the buckle transversely to the trailing region of the strap.

FIG. 3 shows the leading end of tie device 22, particularly needle 32 and a leading end of strap 34 to which the needle is attached, either directly or indirectly. The needle and the strap may be provided by components that are discrete from one another. For example, the strap may be formed of a polymer and the needle may be formed of metal. In some embodiments, the trailing end of the needle may be attached to a suture (e.g., a wire), which in turn is attached to the strap. In some embodiments, the needle and the strap may be formed by the same material and the same piece, such that the needle and strap are continuous with one another.

Strap 34 may form a neck portion 110 that provides a terminus of leading region 40. The neck portion is narrower than other regions of the strap and may be sized to be received in a bore 112 formed at the trailing end of needle 32. The needle then may be crimped at the trailing end to secure the needle to the neck portion. In other examples, the needle may be attached to the strap with an adhesive, a fastener, threaded engagement, a snap-fit, press fitting, clipped into place, by embedding in the leading end of the strap (such as by molding the leading end of the strap around the trailing end of the needle), or the like.

Needle 32 may have any suitable properties. The needle may be formed by any sharp implement or portion of the tie device that is rigid enough to be driven through soft tissue and/or bone, to form a path for the strap around and/or through bone. Generally, the needle is equipped with a sharp tip 114, and is substantially less compliant than the strap (i.e., substantially more rigid). The needle may be rendered less compliant than the strap by forming the needle of a different material and/or by making the needle thicker than the strap. The needle may be curved (as shown here) or straight, among others. If curved, the needle may correspond to any suitable portion of a circle, such as about one-fourth, one-third, or one-half, among others.

FIGS. 4 and 5 show exemplary cross-sectional shapes of the needle. The cross-section may be circular (FIG. 4), triangular (FIG. 5), oval, or the like. Furthermore, the cross-sectional shape may change along the needle, as shown here. In some embodiments, the maximum width of the needle may be at least about as wide as the average or maximum width of the strap, such that the needle forms a path of sufficient width around bone for receiving the strap. Alternatively, the maximum width of the needle may be less than the average or maximum width of the strap, such that the strap widens the path formed by the needle.

FIG. 6 shows an exemplary cross-sectional shape of the strap. The strap in cross-section may, for example, be at least generally rectangular, as shown here, or oval, circular, square, or the like. The cross-sectional shape may be uniform or may vary along the strap. The upper portion of the strap may be smoothly contoured in cross-section to avoid sharp edges and minimize irritation to adjacent soft tissue.

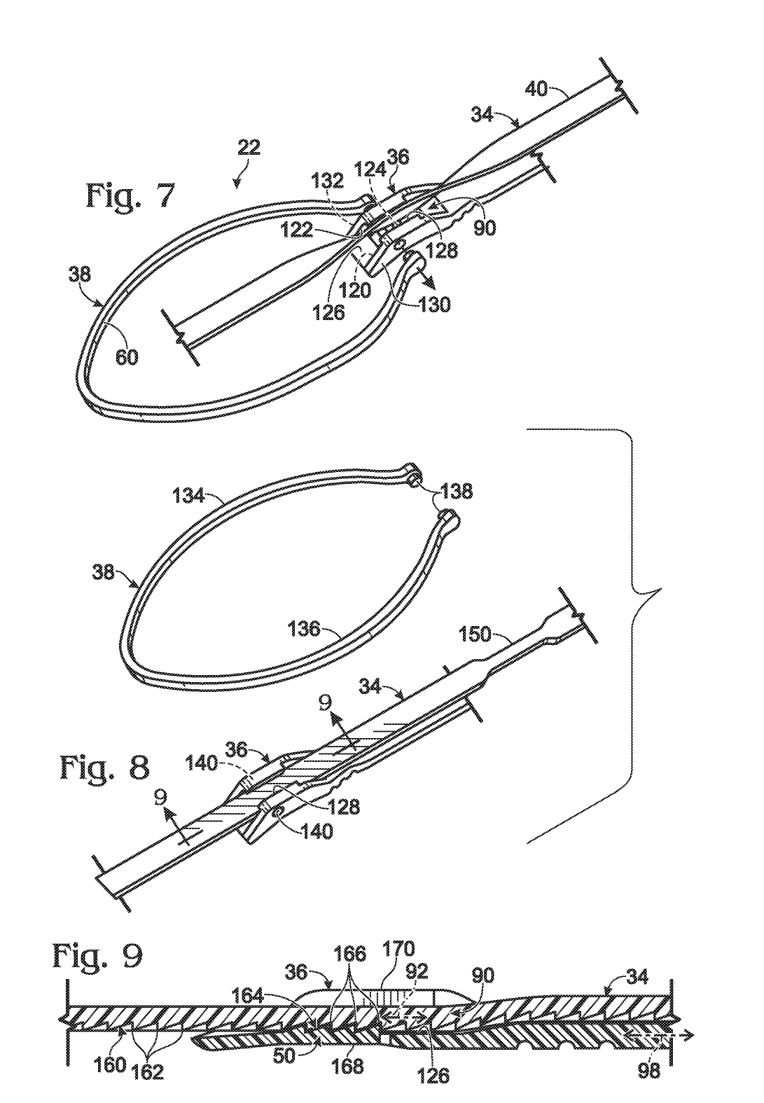

FIG. 7 shows buckle 36 and a handle 38 of cable tie 22 with leading region 40 of strap 34 being placed into channel 90 of buckle 36 from above the buckle, which may be described as top-loading. The strap may be twisted about one-quarter turn, as depicted here, before the strap is loaded into the buckle.

Channel 90 may be bounded by opposing lateral side walls 120, 122, a top wall 124, and a bottom wall 126. The opposing inner side walls may restrict lateral movement of the strap in the channel and may be spaced by a greater distance than the width of the strap, to permit the strap to move along the channel. Each inner side wall (and/or the buckle) may opposingly taper toward the ends of the channel, to minimize the profile of the buckle above bone. Top wall 124 may project from the side walls to restrict upward removal of the strap from the channel. The top wall may define a mouth or entry site 128 of channel 90 for top-loading. Mouth 128 extends from the entrance end to the exit end of the channel.

Channel 90 may be accessible from a transverse direction (e.g., via mouth 128), to permit leading region 40 of the strap to be operatively disposed in the channel by motion of the leading region transverse to travel axis 92 (see FIG. 2), such as from above or from the side of the buckle, among others. In other words, strap 34 may be placed into buckle channel 90 through a top opening and/or a side opening of the buckle, instead of or in addition to an end opening of the buckle.

Buckle handle 38 may be attached to buckle 36 at one or more attachment sites. For example, as shown here, the buckle handle may be connected to the buckle at two spaced sites, namely, at both opposing outer surface regions 130, 132 of the lateral side walls, to form loop 60.

The attachment sites may be created during or after formation of the buckle and/or buckle handle. In some embodiments, the attachment sites may be created by forming the buckle and buckle handle from the same piece of material, such as by molding. Alternatively, the buckle handle may be attached to a pre-formed buckle using an adhesive, by bonding, with a discrete fastener(s), by complementary mating structure, or the like. For example, FIGS. 7 and 8 show handle 38 as a separate piece that can be pre-mated with the buckle and that can be detached from the buckle manually. In particular, legs 134, 136 of handle 38 may be detached from the buckle by urging the legs apart from one another, to remove pins 138 of the legs from sockets 140 defined by the buckle (or vice versa). In any event, the buckle handle may be connected to the buckle in any manner that permits removal of the handle from the cable tie following placement of the cable tie around bone.

FIG. 8 shows another embodiment of strap 34 that includes a neck portion 150. The neck portion may be sized to be placed through mouth 128 of the buckle and into the buckle channel. Accordingly, neck portion 150 may be longer and narrower than mouth 128. The neck portion may (or may not) be flanked longitudinally in one or both opposing directions by regions of the strap that are wider than the neck portion, and that cannot fit through mouth 128. With this configuration, a leading end of the cable tie ahead of the neck portion can be structured with greater design flexibility because there is no requirement that the leading end pass through the buckle during cable tie assembly around bone. For example, it may be difficult to pass a curved needle through buckle 36. Accordingly, the top-loading configuration shown here may save time during surgery, relative to an end-loading configuration, because only a shorter section of the strap needs to pass through the buckle to reach a tensioned configuration.

### III. EXEMPLARY RATCHET MECHANISM

FIG. 9 shows ratchet mechanism 50 formed collectively by strap 34 and buckle 36. The strap may provide a rack 160 formed of strap teeth 162, and the buckle may provide a pawl 164 formed of one or more buckle teeth 166.

The teeth of the strap and buckle may be formed on and/or in any suitable surface(s) of the strap and buckle. In exemplary embodiments, the strap teeth are formed on and/or in an inner surface of the strap and are arranged along any suitable portion or all of the leading (and/or trailing) region of the strap. The buckle teeth mate with the strap teeth.

Pawl 164 may be formed by bottom wall 126. In some embodiments, the pawl may be provided by a cantilevered tab or tongue 168 supported by a frame 170 of the buckle. The tongue may, for example, be formed below channel 90 and may have a flexible connection to the frame that permits the tongue to pivot/move slightly, to allow tooth disengagement, as the strap is being advanced through the buckle. Alternatively, or in addition, the buckle teeth and/or tongue may be formed by one or both of the lateral side walls and/or the top wall of the buckle.

In any event, the buckle teeth and the strap teeth may be configured to engage one another in a successive series of registrations as the strap is pulled through the buckle. In each engaged configuration, withdrawal motion of the strap is restricted by the teeth, while advancement motion is permitted. More generally, rack 160 of strap 34 may engage buckle 36 in channel 90 to permit tightening advancement of the strap along the channel, but not loosening withdrawal of the strap from the channel.

The buckle teeth and the strap teeth may have any suitable structure. For example, the buckle teeth and/or the strap teeth may have a uniform spacing within each set of teeth, and the spacing of the buckle teeth may be the same as or at least correspond to the spacing of the strap teeth. Also, each buckle tooth and/or each strap tooth may have an asymmetrical structure. The leading face of each buckle tooth and the trailing face of each strap tooth may be at least substantially orthogonal to travel axis 92 and/or longitudinal axis 98, when such teeth are mated with one another in the channel. Also, the trailing face of each buckle tooth and the leading face of each strap tooth may be oriented obliquely, such as extending at about the same angle relative to one another and/or at about 10 to 30 degrees from parallel to the travel axis. Stated differently, each tooth may be angled: the buckle teeth may be angled away from the entrance end of channel 90, and the strap teeth may be angled toward the entrance end of channel 90 when mated with the buckle teeth. In any event, the teeth may be disposed in locking engagement that restricts loosening withdrawal of the strap from the buckle.

### IV. EXEMPLARY TIE DEVICE WITH A BREAK-OFF HANDLE AND NEEDLE

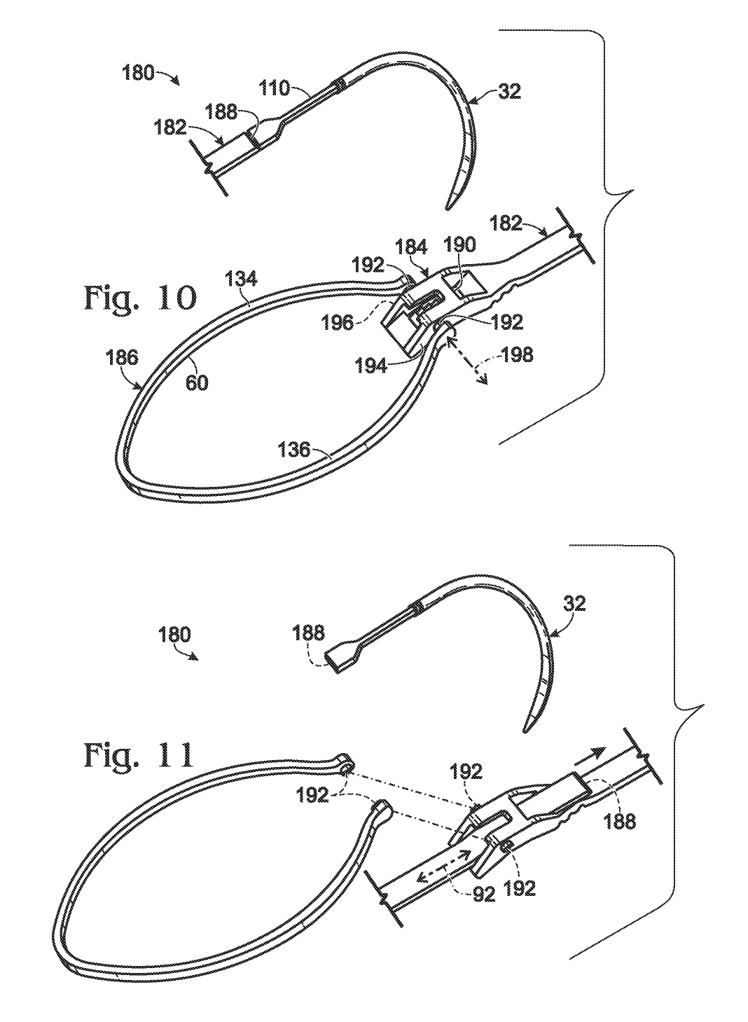

FIGS. 10 and 11 show an exemplary tie device or cable tie 180 with break-off elements. Cable tie 180 may be structured generally as described elsewhere in the present disclosure, such as for cable tie 22 (see FIGS. 1 to 9). The cable tie may be equipped with needle 32, a strap 182, a buckle 184, and a buckle handle 186.

Strap 182 may be similar to strap 34 of cable tie 22, but may include a predefined frangible site 188 at which the strap may be selectively broken by a surgeon. Site 188 may be formed by any feature of the strap that locally decreases the cross-sectional area and/or strength of the strap, such as a transverse groove, opposing notches, or the like. In some embodiments, site 188 may be disposed in and/or correspond to neck portion 110. In some embodiments, the strap may not have a predefined frangible site, and a surgeon may choose a suitable position along the strap where the strap is to be broken or cut, to detach needle 32 from cable tie 180.

Buckle 184 may be similar to buckle 36 of cable tie 22, but may lack mouth 128 for top-loading (e.g., compare FIGS. 7 and 10). In particular, buckle 184 may include a spanning member 190 that obstructs placement of strap 182 (e.g., neck portion 110) into the buckle from above with needle 32 attached. Accordingly, strap 182 is end-loaded into buckle 184 along travel axis 92, after needle 32 has been used to place strap 182 around bone and after the needle is separated from the cable tie (see FIG. 11).

Buckle handle 186 may be similar to buckle handle 38 of cable tie 22, but may be continuous with buckle 184. Handle 186 may form a pair of spaced, frangible attachment sites 192 adjacent opposing outer surface regions of lateral side walls 194, 196 of the buckle. The handle may be broken selectively at sites 192, to detach the handle from the buckle. Breakage of each frangible site may be performed by moving at least a portion of the buckle handle relative to the buckle, such as by pivoting the buckle handle about a transverse axis 198. In some embodiments, each leg 134, 136 of handle loop 60 may be broken off from buckle 184 by twisting or pulling the leg individually. In other cases, a cutting tool may be used to separate the handle from the buckle.

### V. TENSIONING DEVICE FOR A CABLE TIE

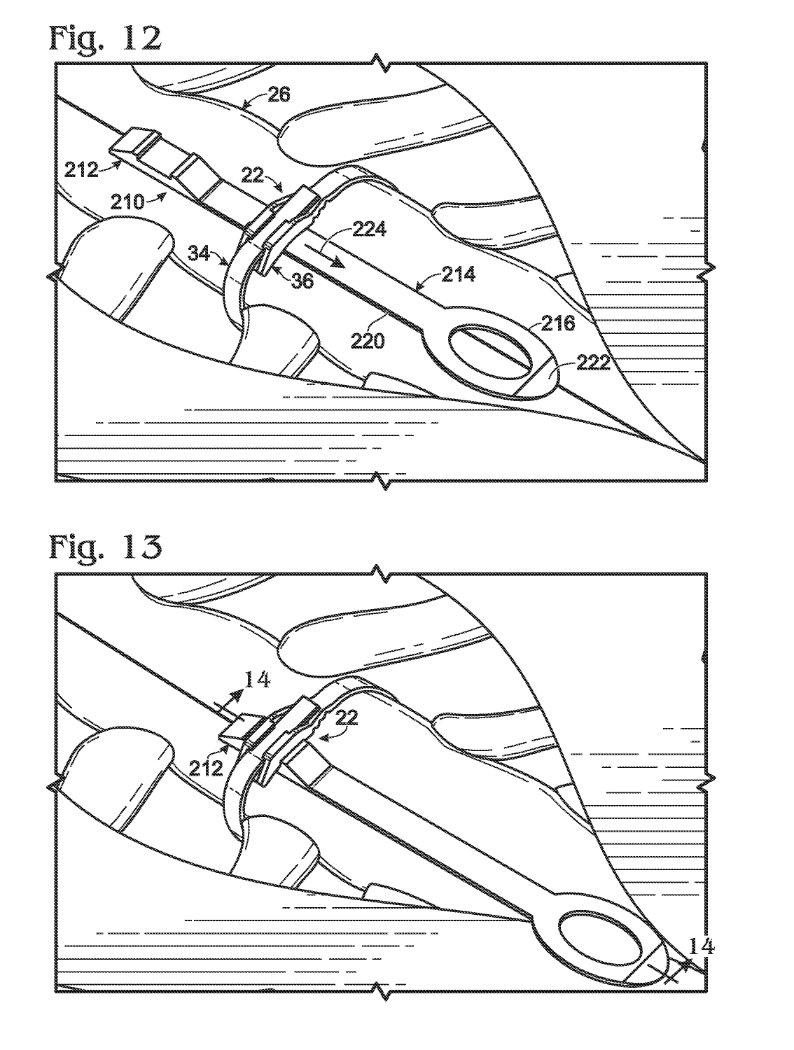

FIGS. 12 and 13 show cable tie 22 assembled around a cut sternum 26, with needle 32 (e.g., see FIG. 1) removed, and with a tensioning device 210 readied to urge a spacer member 212 under buckle 36 (FIG. 12) or with the spacer installed under the buckle (FIG. 13). The tensioning device may be utilized by a surgeon to increase the tension on strap 34, which may, for example, force additional compression of bone. In particular, device 210 may permit installation of spacer 212 under buckle 36 and/or strap 34, to elevate a region of the cable tie from bone.

The spacer may include a wedge region and may be described as a wedge member. The wedge region may provide additional compression of bone beyond the compression a surgeon is capable of effecting by pulling on the strap/handle of the cable tie.

Device 210 may include a handle 214 connected to spacer 212. Handle 214 may include a head 216, such as a finger loop, connected to a tether 220. The tether may be any structure, such as a band of material, a rod, a wire, a cord, or the like, that is capable of transmitting an axially directed force to spacer 212. In some embodiments, head 216 may be omitted, and the tether may be gripped with a gripping tool, such as a needle driver, or by hand. The tether may (or may not) be flexible.

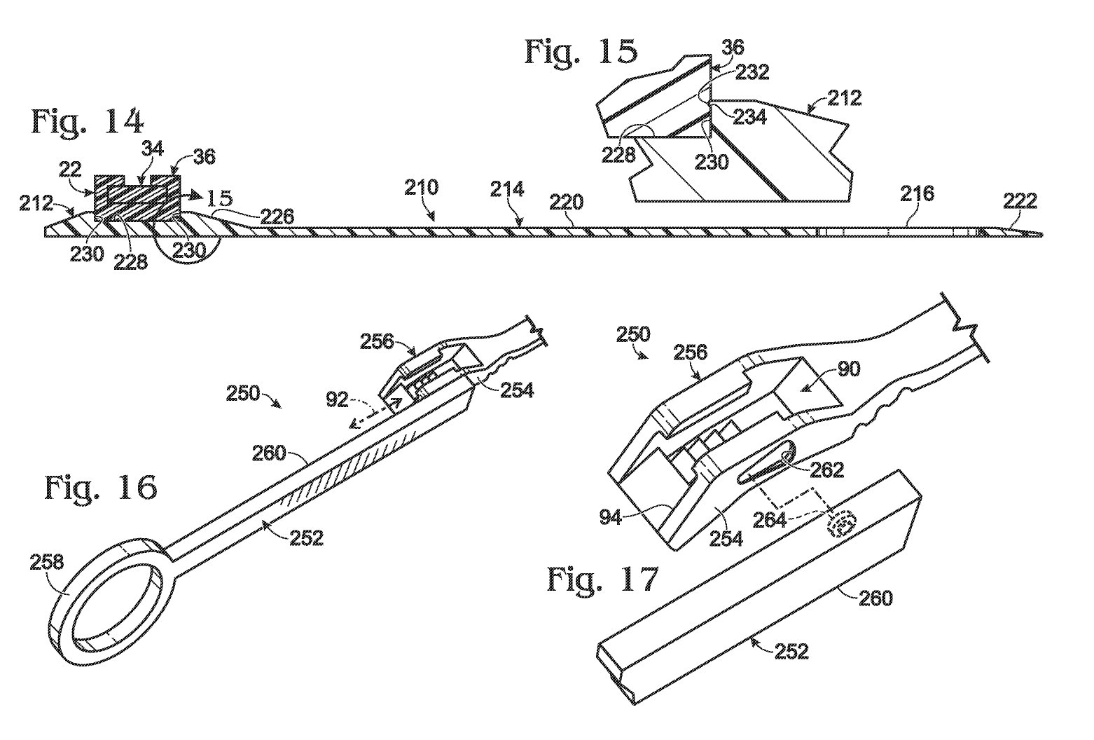

Device 210 may be placed in the position shown in FIG. 12 before or after the cable tie is installed around bone, the strap assembled with the buckle to form a loop, and/or part of the strap removed from the cable tie. Placement of device 210 before pre-tensioning the cable tie may make it easier to fit device 210 between the cable tie and bone. In some embodiments, handle 214 may include a bevel 222 (see FIG. 14 also) that facilitates sliding the tensioning device into the position of FIG. 12 after the cable tie has been secured to itself around bone. In any event, the tensioning device may be positioned as shown, with spacer 212 adjacent the cable tie (e.g., disposed lateral to the buckle) and with tether 220 extending transversely to the cable tie.

The surgeon may exert an axial force, indicated by an arrow at 224 in FIG. 12, on device 210, such as by pulling on finger loop 216, to force spacer 212 under the cable tie, to the position shown in FIG. 13. After spacer 212 has moved to its seated position under the cable tie, handle 214 may be removed, such as by cutting or breaking off tether 220.

FIG. 14 shows a sectional view of tensioning device 210 and cable tie 22. Spacer 212 may form a ramp 226 that slopes toward handle 214. When axial force 224 is applied to device 210 (see FIG. 12), strap 34 and/or buckle 36 of the cable tie may travel up ramp 226, which forces the strap/buckle away from the sternum (or other bone).

The upper surface of spacer 212 may define a recess 228 adjacent ramp 226. The recess may be sized and shaped to receive at least a portion of strap 34 and/or buckle 36. For example, the recess may be at least about as wide as the strap/buckle. Accordingly, when the cable tie reaches the top of ramp 226, the strap/buckle may drop into recess 228 and may be retained there by the tension on the strap and/or retainer structure, such as retaining walls 230 that form opposing sides of the recess. The thickness of the spacer at recess 228 determines how much the path length around bone is increased by spacer installation and thus determines how much the tension on the cable tie is increased.

FIG. 15 shows engaged regions of buckle 36 and spacer 212. The buckle and spacer may include at least one projecting detent 232 that is received in a corresponding hollow 234, to restrict movement of the buckle in the recess.

### VI. METHODS AND KITS

The system disclosed herein provides methods of stabilizing bone. The methods may include any suitable combination of the steps disclosed below in this section and/or elsewhere in the present disclosure. The steps may be performed in any suitable order and each step may be performed any suitable number of times.

A cable tie may be provided and/or selected. The cable tie may include any combination of the features disclosed herein. For example, the cable tie may incorporate any combination of an elongate strap having opposing leading and trailing regions, a surgical needle connected to the leading region, a buckle connected to the trailing region and defining a channel, and a handle connected to the buckle. The handle may be longer than the buckle.

A leading region of the strap may be placed through the buckle such that the cable tie encircles bone. In other words, the leading region may be assembled with the buckle to form a loop. The buckle may be loaded with the strap from an end or a side of the buckle's channel. For example, the strap may be loaded into the buckle from above the buckle. Encircling bone may include encircling a bone and/or bone portions, such as bone portions (e.g., bone fragments) separated by a fracture and/or a cut, among others.

The leading region of the strap may be pulled to decrease the size of the loop and/or to tension the strap around the bone, which may compress bone. For example, the leading region and the handle may be pulled in respective opposing directions. In some examples, pulling may include applying force to the handle via at least one finger of a person disposed in a loop of the handle. (Engagement of the cable tie with a person's hand or a finger(s) thereof includes engagement of the cable tie with a gloved hand/finger.) In some embodiments, the strap may include a strap handle that is engaged manually and/or by a tool to facilitate pulling the leading end of the strap. The strap handle may be formed as a loop in a leading region of the strap. Pulling may include applying force to the strap handle via at least one finger of a person disposed in a loop formed by the strap handle. In some embodiments, pulling may include applying force to the leading region of the strap, the buckle handle, or both, via one or more clamp devices engaged with the cable tie.

The strap may be cut (e.g., severed) to remove at least a portion of the leading region. Cutting the strap may shorten the strap to remove excess length, may separate the needle from the cable tie, and/or may minimize the length of strap extending from the loop encircling bone.

The handle may be detached from the cable tie after the step of pulling. In some cases, the handle and the buckle may be monolithic with one another, and the step of detaching may include breaking a bridge between the handle and the buckle. For example, at least a portion of the handle may be pivoted with respect to the buckle to break off the handle from the buckle. Detachment of the handle may be performed manually without the use of tools or may be performed with a gripping tool and/or a cutting tool, among others. In some cases, the handle may be connected to the buckle via a pair of spaced bridges, and detachment may be performed by breaking and/or cutting each of the bridges.

The cable tie may be tensioned further using a tension augmentation device including a spacer member. The spacer member may define a recess sized to receive a portion of the cable tie. The spacer member may be placed between the bone and the cable tie, with a portion of the buckle and/or strap in the recess, such that tension on the strap is increased. The spacer member may include a ramp, and the spacer member may be urged transversely to the cable tie, to wedge the spacer member between the cable tie and bone, such that the buckle/strap moves up the ramp to lift part of the cable tie away from bone. A portion of the cable tie may be received in the recess, to restrict movement of the cable tie.

Any suitable combination of the system components disclosed herein may be provided as a kit. For example, the kit may include any combination of the following: at least one cable tie, a tensioning device including a spacer member, one or more gripping tools to grip the strap (and/or buckle or handle), a cutting tool to cut the strap (and/or handle or tensioning device), a saw to cut bone, a caliper to measure bone, and the like. In some embodiments, the kit may include a plurality of cable ties of different length, different strap width, and/or different needle size/shape, among others. In some embodiments, the kit may include a plurality of tensioning devices equipped with respective spacer members of different thickness/height.

### VII. CABLE TIE CONSTRUCTION

The cable ties and/or accessories therefor may be constructed of any suitable biocompatible material and may be formed of any suitable number of discrete pieces.

Exemplary biocompatible materials for construction of the cable ties and/or accessories may include (1) a plastic/polymer (for example, ultra-high molecular weight polyethylene (UHMWPE), polymethylmethacrylate (PMMA), polytetrafluoroethylene (PTFE), polyetheretherketone (PEEK), nylon, polypropylene, and/or PMMA/polyhydroxyethylmethacrylate (PHEMA)); (2) a bioresorbable (bioabsorbable) material or polymer (for example, polymers of α-hydroxy carboxylic acids (e.g., polylactic acid (such as PLLA, PDLLA, and/or PDLA), polyglycolic acid, lactide/glycolide copolymers, etc.), polydioxanones, polycaprolactones, polytrimethylene carbonate, polyethylene oxide, poly-β-hydroxybutyrate, poly-β-hydroxypropionate, poly-δ-valerolactone, other bioresorbable polyesters, etc.; (3) a composite (e.g., a polymer matrix (such as PEEK) containing carbon fibers and/or ceramic); (4) metal (for example, titanium or titanium alloys, alloys with cobalt and chromium (cobalt-chrome), stainless steel, etc.); and/or the like.

The material(s) composing a cable tie may be selected based on various considerations. For example, the material may confer sufficient flexibility for a strap and/or buckle of the cable tie, to allow the strap to be wrapped around bone and the buckle to permit the strap to be engaged in various registrations. Also, or alternatively, the material(s) may be picked based on whether the cable tie is a one-piece or multi-piece (e.g., two-part or three-part) construct.

A cable tie, and particularly a strap, buckle, and/or handle(s) thereof, may be constructed as a unitary or continuous article, that is, as only one piece. For example, the cable tie, or any suitable portion thereof, may be monolithically formed, such as by molding the cable tie from a polymer.

Alternatively, the cable tie may be composed of two or more discrete pieces, which may be formed of the same or different materials. If formed as two or more discrete pieces, the pieces may be attached to one another by any suitable mechanism, such as an adhesive, bonding, a press fit, mating of complementary structure, molding one component in engagement with another, or the like. In some embodiments, the cable tie may be composed of a strap and a buckle formed separately from each other. In some embodiments, the cable tie may be composed of a handle formed separately from a strap and buckle. The strap and buckle (or handle and strap/buckle) may be formed of different materials. For example, the strap may be formed of a more compliant material than the buckle, to conform the strap more effectively to the surface contours of a bone, while the buckle may be formed of a less compliant material, to provide a more secure connection of the buckle to the strap. For example, the strap may be composed of a polymer(s) and the buckle of metal (e.g., titanium or a titanium alloy). In this way, the buckle may be very strong and resistant to deformation (e.g., in the top-loading embodiment), while maintaining a low profile. PEEK may be a more desirable polymer for the strap than some other polymers (such as nylon), because PEEK is more resistant to creep (gradual stretching) under load. As another example, the buckle and strap may be formed of a bioresorbable material, while the handle, which may be removable from the buckle and strap, may be formed of a non-bioresorbable material, which may be selected to give the handle greater strength and/or rigidity, among others. In some embodiments, the cable tie may include a needle formed of metal and a strap, buckle, and/or handle formed of at least one polymer.

### VIII. EXAMPLES

The following examples describe additional aspects of exemplary surgical cable ties. These examples are intended for illustration and should not limit the entire scope of the present disclosure.

### Example 1

### Exemplary Cable Tie with Asymmetrically-Connected Handle

This example describes an exemplary cable tie 250 with a handle 252 that is attached to only one outer surface region of a side wall 254 of a buckle 256; see FIGS. 16 and 17. The strap of tie 250 is not passed through a loop formed by the handle (and buckle) before loading the strap into the buckle (in contrast to cable tie 22; see FIG. 1). Accordingly, with the handle design of cable tie 250, the surgeon does not need to release and re-engage the strap/needle when passing the needle and strap through the handle, as is generally necessary when the strap is passed through a loop.

Buckle handle 252 may include a head 258 and a stem 260 that connects the head to the buckle. Head 258 may be structured as a loop (e.g., a finger loop), as shown here, or any other structure that facilitates manual engagement and/or tool engagement (e.g., with a gripping tool). Stem 260 may extend at least substantially parallel (e.g., within about 30, 20, or 10 degrees from parallel) to travel axis 92 defined by buckle 256.

Stem 260 may be keyed to buckle 256. The buckle may define a slot 262 that receives a projection 264 formed on the stem (or vice versa). The slot may narrow toward an entrance end 94 of channel 90, such that projection 264 cannot be removed from the slot when the handle is being pulled to tension the cable tie. The handle may be detached from the buckle after use by moving projection 264 to the wider end of the slot. In other embodiments, detachment of the stem may be performed by breaking or cutting the stem off of the buckle.

### Example 2

### Exemplary Tie Device with Prongs

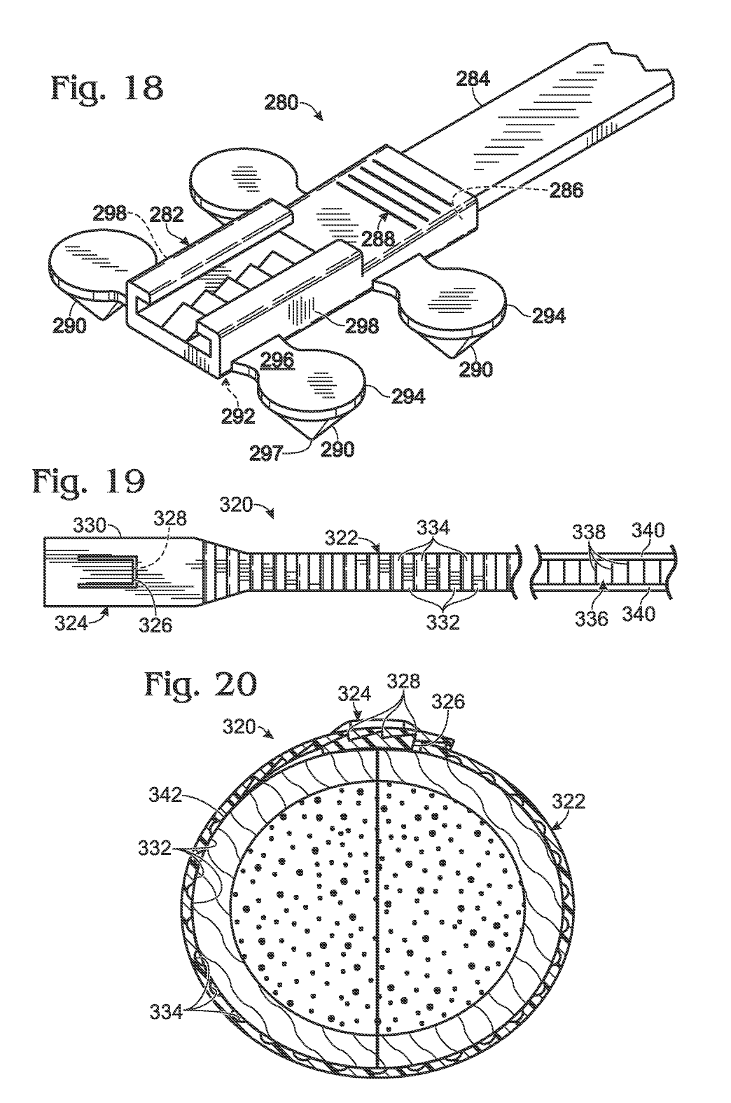

This example describes an exemplary cable tie 280 including prongs and having a buckle 282 and a strap 284 that are discrete from each other; see FIG. 18.

Buckle 282 may define a socket 286 that receives an end of strap 284. The buckle may be crimped, shown at 288, adjacent the socket, to secure the buckle and strap to each other. The strap may, for example, be formed of plastic and the buckle of metal, both may be formed of plastic, or both may be formed of metal, among others. In other embodiments, the strap and buckle may be continuous with each other.

Cable tie 280 may include one or more prongs 290 formed on an inner surface 292 of the tie and configured to engage bone, to resist slippage of the cable tie on bone. The tie may have one or more prongs formed on buckle 282, one or more prongs formed on strap 284, one or more prongs formed on one or more feet or tabs 294 that project from the buckle and/or strap, or a combination thereof, among others. If formed on the strap, the prongs may be disposed on the trailing region of the strap (or on one or more feet projecting from the trailing region), such as adjacent the buckle.

Each prong is a pointed projection. The pointed projection may have any suitable first dimension, as measured from inner surface 292 in the direction in which the prong projects (e.g., orthogonally to inner surface 292), and any suitable second dimension, as measured at the base of the prong where the prong meets inner surface 292. For example, the first dimension may be less than the second dimension (a short prong), about the same as the second dimension, or greater than the second dimension (a long prong). Also, the first dimension may be less than, about the same as, or greater than the buckle, strap, or foot height, as measured between inner surface 292 and an outer surface 296 of the buckle, strap, or foot. The prong may have any suitable cross-sectional shape, such as circular, oval, triangular, square, rectangular, hexagonal, or the like. The cross-sectional shape may be constant or may change as the prong projects to its tip 297. In some examples, the prong may have a substantially linear taper or may have an increasing taper (a tip that is convex in profile) or decreasing taper (a tip that is concave in profile) as the prong extends to tip 297. In some examples, the prong may be at least generally conical in shape.

Each foot 294 may project laterally from the buckle and/or strap. Accordingly, the foot may increase the footprint of the cable tie on bone and may facilitate distributing the load over a greater surface area of bone. In some cases, the buckle may include one or more feet 294 (such as a pair of feet) projecting from each lateral side 298 of the buckle.

The prong or prongs of the cable tie, after tie installation, may be disposed on only one side of a discontinuity in bone (e.g., in engagement with only one sternal fragment/half) or at least one prong or at least a pair of prongs may be disposed on each side of the discontinuity (e.g., in engagement with each sternal fragment/half).

### Example 3

### Exemplary Tie Device with Ridges to Improve Blood Flow

This example describes an exemplary cable tie 320 with a strap 322 designed to reduce the amount of surface area of bone that the tie contacts; see FIGS. 19 and 20.

Tie 320 includes a buckle 324 attached to strap 322. Buckle 324 may include any of the features disclosed herein, such as a cantilevered tongue 326 providing a pawl 328 and supported by a frame 330.

The inner surface of strap 322 may include different structural features arranged along the strap. The strap may have a trailing region with a plurality of transverse ridges 332 and grooves 334 arranged in alternation along the strap. Each ridge and groove may extend at least substantially completely across the strap, that is, from edge to edge of the strap. The strap also may have a leading region forming a rack 336 with teeth 338 that can engage pawl 328. Teeth 328 may be flanked laterally on both sides by a border strip 340 such that the teeth are recessed.

FIG. 20 shows tie 320 secured around a cut bone 342. Ridges 332 contact the surface of the bone and grooves 334 are spaced from the bone. In this way, damage to the periosteum is reduced and there is less negative impact on blood flow. The ridges and grooves may be arranged along any suitable length of the strap, such as a length that is less than the average or minimum circumference of the type of bone to be stabilized. For example, here, the trailing region of the strap with its ridges and grooves extends around more than three-fourths of the bone circumference. The overall length of the strap is generally greater than the maximum circumference to the type of bone to be stabilized.

### Example 4

### Selected Embodiments

This example describes selected embodiments of the present disclosure, presented as a series of indexed paragraphs.

1. A tie device for stabilizing bone, comprising: (A) a strap including a leading region and a trailing region; (B) a needle connected to the leading region of the strap; (C) a buckle connected to the trailing region of the strap and configured to receive the leading region of the strap such that the buckle and the strap collectively form a loop and a ratchet mechanism; and (D) at least one prong formed on from an inner surface of the tie device and configured to engage bone.

2. The tie device of paragraph 1, wherein the at least one prong includes a plurality of prongs.

3. The tie device of paragraph 1 or 2, wherein the at least one prong includes a prong having a first dimension measured in a direction in which the prong projects from the inner surface and a second dimension measured at a base of the prong where the prong meets the inner surface, and wherein the first dimension is less than the second dimension.

4. The tie device of paragraph 1 or 2, wherein the at least one prong includes a prong having a first dimension measured in a direction in which the prong projects from the inner surface and a second dimension measured at a base of the prong where the prong meets the inner surface, and wherein the first dimension is greater than the second dimension.

5. The tie device of any preceding paragraph, wherein the at least one prong include a prong formed on the strap.

6. The tie device of any preceding paragraph, wherein the at least one prong includes a prong formed on the buckle.

7. The tie device of any preceding paragraph, wherein the at least one prong includes a prong formed on a foot that projects laterally from the strap.

8. The tie device of any preceding paragraph, wherein the at least one prong includes a prong formed on a foot that projects laterally from the buckle.

9. The tie device of any preceding paragraph, wherein the at least one prong includes a pair of prongs formed on a pair of feet that project from opposing sides of the buckle.

10. The tie device of any preceding paragraph, wherein the at least one prong includes a first pair of prongs formed on a first pair of feet that project from a first opposing side of the buckle and a second pair of prongs formed on a second pair of feet that project from a second opposing side of the buckle.

11. The tie device of any preceding paragraph, wherein the at least one prong includes a prong having a circular or oval cross-section.

12. The tie device of any preceding paragraph, wherein the at least one prong includes a prong having a polygonal cross-section.

13. The tie device of any preceding paragraph, wherein the buckle is metal and the strap is plastic.

14. The tie device of any preceding paragraph, wherein the buckle or the strap is continuous with the at least one prong.

15. The tie device of any preceding paragraph, wherein the ratchet mechanism permits advancement of the leading region from the buckle in a first direction that tightens the loop and restricts withdrawal of the leading region from the buckle in a second direction that loosens the loop, further comprising a handle connected to the buckle and extending away from the buckle, thereby allowing a person to apply respective opposing forces to the leading region of the strap and to the buckle via the handle, to tension the loop around bone.

16. A method of stabilizing bone, the method comprising: (A) providing the tie device of any preceding paragraph; and (B) installing the tie device in a tensioned configuration around bone.

17. The method of paragraph 16, wherein the step of installing includes a step of engaging the bone with the at least one prong.

18. The method of paragraph 17, wherein the step of engaging includes a step of engaging a sternum.

19. The method of any of paragraphs 16 to 18, further comprising a step of cutting the bone.

20. The method of any of paragraphs 17 to 19, wherein the step of engaging the bone includes a step of engaging the bone with a pair of prongs disposed on opposing sides of a discontinuity in the bone.

21. The method of any of paragraphs 17 to 19, wherein the step of engaging the bone includes a step of engaging the bone with the at least one prong on only side of a discontinuity in the bone.

22. The method of any of paragraphs 17 to 21, wherein the step of engaging the bone includes a step of penetrating a surface of the bone with a prong.

23. The method of paragraph 22, wherein the step of penetrating a surface of the bone with a prong includes penetrating the periosteum of the bone.

24. A tie device for stabilizing bone, comprising: (A) a strap including a leading region and a trailing region; (B) a needle connected to the leading region of the strap; (C) a buckle connected to the trailing region of the strap and configured to receive the leading region of the strap such that the buckle and the strap collectively form a loop and a ratchet mechanism; wherein the strap includes a neck portion, wherein the buckle defines a travel axis through the buckle for advancement of the strap, and wherein the buckle is capable of receiving the neck portion into coaxial alignment with the travel axis from a direction transverse to the travel axis.

25. The tie device of paragraph 24, wherein the buckle defines a passage for advancement of the strap along the travel axis, and wherein the neck portion is receivable in the passage from above the buckle.

26. The tie device of paragraph 24 or 25, wherein the buckle defines a passage for advancement of the strap along the travel axis and a mouth that permits the neck portion of the strap to be received in the passage from the direction transverse to the travel axis.

27. The tie device of any of paragraphs 24 to 26, wherein the strap is wider than the neck portion in both opposing directions along the strap from the neck portion.

28. A system for stabilizing bone, comprising: (A) a tie device including (i) a strap including a leading region and a trailing region, (ii) a needle connected to the leading region of the strap, and (iii) a buckle connected to the trailing region of the strap and configured to receive the leading region of the strap such that the buckle and the strap collectively form a loop and a ratchet mechanism; and (B) a tensioning device including a spacer member configured to be placed under the tie device while the tie device is looped around bone, such that the spacer member increases the tension of the strap.

29. The system of paragraph 28, wherein the spacer member defines a recess sized to receive a portion of the tie device.

30. The system of paragraph 29, wherein the spacer member defines a recess sized to receive a portion of the strap.

31. The system of paragraph 29, wherein the spacer member defines a recess sized to receive a portion of the buckle.

32. The system of any of paragraphs 28 to 31, wherein the spacer member is configured to be wedged between the tie device and bone.

33. The system of any of paragraphs 28 to 32, wherein the tensioning device includes a handle portion connected to the spacer member.

34. The system of paragraph 33, wherein the handle portion includes a loop sized to receive at least one finger of a person.

35. The system of paragraph 33 or 34, wherein the spacer member is connected to the handle portion by a band of material.

36. A method of stabilizing bone, the method comprising: (A) providing the system of any of paragraphs 28 to 35; and (B) placing the spacer member between the bone and the tie device such that tension on the strap is increased.

37. The method of paragraph 36, wherein the spacer member includes a ramp, and wherein the step of placing includes a step of urging the spacer member transversely to the strap, such that part of the tie device moves up the ramp.

38. The method of paragraph 36 or 37, wherein the step of placing the spacer member includes a step of wedging the spacer member between the tie device and bone.

39. The method of any of paragraphs 36 to 38, further comprising a step of detaching a portion of the tensioning device after the step of placing.

40. The method of paragraph 39, wherein the step of detaching includes a step of detaching a handle portion of the tensioning device.

41. The method of paragraph 39 or 40, wherein the step of detaching is performed by cutting or breaking the tensioning device.

42. The method of any of paragraphs 39 to 41, wherein the step of placing includes a step of pulling the tensioning device.

43. The method of paragraph 42, wherein the tensioning device defines a long axis, wherein the step of pulling includes a step of applying tension to the tensioning device in a direction substantially parallel to the long axis.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure. Further, ordinal indicators, such as first, second, or third, for identified elements are used to distinguish between the elements, and do not indicate a particular position or order of such elements, unless otherwise specifically stated.

1. A tie device for stabilizing bone, comprising:
a strap including a leading region and a trailing region;
a needle connected to the leading region of the strap;
a buckle connected to the trailing region of the strap and configured to receive the leading region of the strap such that the buckle and the strap collectively form a loop and a ratchet mechanism, the ratchet mechanism permitting advancement of the leading region from the buckle in a first direction that tightens the loop and restricting withdrawal of the leading region from the buckle in a second direction that loosens the loop; and
a handle connected to the buckle and extending away from the buckle at least generally in the second direction, thereby allowing a person to apply respective opposing forces to the leading region of the strap and to the buckle via the handle, to tension the loop around bone.

2. The tie device of claim 1, wherein the loop is a first loop, and wherein the handle alone or in combination with the buckle forms a second loop sized to receive at least one finger of a person.

3. The tie device of claim 1, wherein the buckle includes a pair of side walls, and wherein the handle connects to the buckle at one or both of the side walls.

4. The tie device of claim 1, wherein the handle is configured to be detached from the buckle at one or more predefined sites.

5. The tie device of claim 1, wherein the handle and the buckle are continuous with one another.

6. The tie device of claim 5, wherein the handle and the buckle are connected by at least one predefined frangible site configured to be selectively broken to separate the handle from the buckle.

7. The tie device of claim 1, wherein the buckle defines a channel that receives the strap to actuate the ratchet mechanism, where the channel defines a travel axis for travel of the strap through the buckle, and wherein the travel axis is substantially parallel to the first and second directions.

8. The tie device of claim 1, wherein the handle is a first handle, further comprising a second handle formed as a longitudinal slit in the strap.

9. The tie device of claim 1, wherein the buckle is formed of metal and the strap of plastic.

10. The tie device of claim 1, wherein the strap includes a neck portion, wherein the buckle defines a travel axis through the buckle for advancement of the strap, and wherein the buckle is capable of receiving the neck portion into coaxial alignment with the travel axis from a direction transverse to the travel axis.

11. The tie device of claim 1, wherein the buckle includes one or more cleats configured to reduce slippage of the buckle on bone by engaging bone.

12. A method of stabilizing bone, comprising:
selecting a tie device including a strap with a leading region and a trailing region, a needle connected to the leading region, a buckle connected to the trailing region, and a handle connected to the buckle;
placing the strap in the buckle such that the tie device forms a loop around bone and the strap and buckle collectively form a ratchet mechanism restricting withdrawal of the strap from the buckle;
pulling the strap and the handle in at least generally opposing directions to tighten the loop around bone; and
detaching the handle from the buckle.

13. The method of claim 12, wherein the step of detaching includes a step of breaking or cutting the handle adjacent the buckle.

14. The method of claim 12, wherein the step of detaching includes a step of separating a buckle and a handle that are discrete from each other.

15. The method of claim 12, wherein the handle is connected to the buckle at a pair of spaced attachment sites, and wherein the step of detaching includes a step detaching the strap from the buckle at each of the attachment sites.

16. The method of claim 12, wherein the handle is connected to only one side of the buckle.

17. The method of claim 12, further comprising a step of installing a spacer member between the tie device and bone to increase the tension of the strap, the spacer member defining a recess sized to receive a region of the buckle, the strap, or both.

18. The method of claim 17, wherein the step of installing includes a step of wedging a ramp of the spacer member between the tie device and bone.

19. The method of claim 12, wherein the strap, the buckle, or both are formed of PEEK.
